(19) United States
(12) Patent Application Publication
(10) Pub. No.: US 2021/0113220 A1
(43) Pub. Date: Apr. 22, 2021

(54) Cranial Drill Guide

(71) Applicant: Ad-Tech Medical Instrument Corp., Oak Creek, WI (US)

(72) Inventors: David A. Putz, Pewaukee, WI (US); Kyle Horton, Seattle, WA (US); Brian Yasosky, Greenfield, WI (US)

(73) Assignee: Ad-Tech Medical Instrument Corp., Oak Creek, WI (US)

(21) Appl. No.: 16/657,120

(22) Filed: Oct. 18, 2019

(51) Int. Cl. A61B 17/17; A61B 17/16

(52) U.S. Cl. CPC A61B 17/1739; A61B 17/1695

(57) ABSTRACT

In an improved cranial drill guide for guiding a drill bit during drilling a brain-access hole in a skull, the improvement comprising an elongate guide-body having a wall defining an internal through-hole therealong, the through-hole sized to receive the drill bit, and a side-opening in the wall for passage of bone debris from the drill bit to outside the guide-body.

### FIELD OF THE INVENTION

The invention relates generally to the field of neurosurgery and more specifically to systems used for accurately drilling a brain-access hole in a patient's skull as part of a neurosurgical procedure.

### BACKGROUND OF THE INVENTION

In the field of neurosurgery, there are many instances in which it is necessary to drill brain-access holes in a patient's skull. Among these types of procedures is the insertion of a depth electrode into a human brain for the monitoring of the electrical signals in the brain. Such signals are called electroencephalograph signals or an EEG. One very important use of such signals is diagnostic mapping of EEG signals in the brain of a patient suffering from intractable epilepsy in order to determine if and where in the brain a subsequent surgical procedure can be carried out to bring relief to the patient.

Recent advances in such surgical procedures have led to the use of surgical robots such as ROSA®, a system provided by Medtech S.A., a French company headquartered in Montpellier, France. Medtech's own description of the system states: “ROSA® acts as a kind of ‘GPS’ for the skull, and may be used for various types of cranial intervention requiring surgical planning based on pre-operative data, precise location of the patient's anatomy and accurate positioning and handling of instruments.” The system is used to assist surgeons in “surgical interventions such as biopsies, electrode implantation for functional procedures (stimulation of the cerebral cortex, deep brain stimulation), open skull surgical procedures requiring a navigation device, endoscopic interventions and any other ‘key-hole’ procedures.”

Other surgical robots are employed by surgeons today. Another system developed for use in brain surgery is the Renaissance® system from Mazor Robotics of Caesarea, Israel.

Various instruments have been employed within robot systems to guide a drill during the drilling of a brain-access hole in a skull. A drill guide with some similarities but without the important and advantageous features of the present invention has been available in the past from Ad-Tech Medical Instrument Corporation of Oak Creek, Wisconsin.

Another drill guide, disclosed in published application EP 3 332 718 by Steven Gill et al. titled “Cranial Drill System,” has a stepped design which includes a drill element for drilling into the skull, after which the guide serves as a sleeve for a drill for the brain tissue.

A drill guide is disclosed in published application WO 2019/099881 by Joshua Bederson titled “Epidural/subdural Minimally Invasive Access Tool.” This drill guide has a housing and a drill guide sleeve assembly attached thereto. The drill guide sleeve assembly has at least two degrees of freedom to allow the drill guide sleeve assembly to be moved to a first pivoted position for forming an angled burr hole through a cranial surface. The two degrees of freedom can comprise a pivoting motion of the drill guide sleeve assembly and an axial motion of a portion of the drill guide sleeve assembly.

A surgical guide tool is disclosed in published application WO 2019/071141 by Wolfgang Fischler titled “Drilling Platform Tool for Surgeries.” The surgical guide tool includes: (a) a platform including one or more supports for body-part attachment; (b) a block with a top and bottom planar surfaces and a guide aperture extending from the top to bottom surface for guiding a surgical instrument in making a drill hole: and (c) an intermediate module removably positioned between the platform and the block.

Other systems and devices are also used in the field of neurosurgery for positioning instruments such as drill guides. Among these are stereotactic frames which mechanically form a three-dimensional coordinate system for precise location of such instruments.

There is a need for a cranial drill guide that, while guiding a drill along much of its length, also provides a way for the bone debris to exit from the drill bit during drilling. Such a device will also eliminate the possibility that the drill bit will seize up during drilling. The cranial drill guide described herein addresses these and other shortcomings of the prior art.

### OBJECTS OF THE INVENTION

It is an object of the present invention to provide a cranial drill guide that, while guiding a drill bit during neurosurgery, enables bone debris from the drilling process to exit through an opening in the side of the drill guide.

Another object of the inventive cranial drill guide is to eliminate instances of the drill bit seizing up in the drill guide due to bone debris.

Another object of the inventive cranial drill guide is to lower the amount of heat generated during the drilling process.

Another object of the present invention is to provide a cranial drill guide which grips the skull at its tip to secure the position of the drill guide during drilling.

Still another object of the present invention is to provide a drill guide which is suitable for use in systems for positioning instruments during neurosurgical procedures.

These and other objects of the invention will be apparent from the following descriptions and from the drawings.

### SUMMARY OF THE INVENTION

The present invention is an improved cranial drill guide for guiding a drill bit during drilling a brain-access hole in a skull. The improved drill guide includes an elongate guide-body having a wall defining an internal through-hole therealong, the through-hole being sized to receive the drill bit, and a side-opening in the wall for passage of bone debris from the drill bit to outside the guide-body.

In some preferred embodiments, the side-opening is positioned adjacent to a distal end of the drill guide.

In some highly-preferred embodiments, the cranial drill guide also includes a serrated tip at a distal end of the guide-body for securing the position of the drill guide with respect to the skull.

Some embodiments of the cranial drill guide of the present invention have a second side-opening such that bone debris can pass out two sides of the guide-body, and in some of these embodiments, the side-openings are elongate slots positioned opposite each other.

Some of the embodiments of the inventive cranial drill guide have a side-opening which is formed by removing substantially one-half of the guide-body cross-section along a portion of the guide-body.

In some preferred embodiments, the cranial drill guide also includes a drill-guide-head at a proximal end of the guide-body which has cross-sectional and length dimensions different from those of the guide-body.

In some preferred embodiments, the drill guide is configured to be held within systems for positioning instruments during neurosurgical procedures.

In some preferred embodiments of the cranial drill guide, the guide-body has a circular cross-section.

### BRIEF DESCRIPTION OF THE DRAWINGS

FIG. 1A is a perspective drawing of an embodiment of the inventive cranial drill guide of this invention.

FIG. 1B is a side elevation drawing of the cranial drill guide of FIG. 1A showing a side-opening in the wall of the guide-body.

FIG. 1C is a side elevation drawing of the cranial drill guide of FIG. 1A showing the side opposite to that of FIG. 1B.

FIG. 1D is a proximal-end elevation drawing of the cranial drill guide of FIG. 1A.

FIG. 2 is a partial orthographic projection drawing of the embodiment of the inventive cranial drill guide of FIG. 1A, showing side and end views and also showing a sectional view and an enlarged view of portions of the cranial drill guide.

FIG. 3A is a perspective drawing of a first alternative embodiment of the inventive cranial drill guide of this invention.

FIG. 3B is a side elevation drawing of the cranial drill guide of FIG. 3A showing a side-opening in the wall of the guide-body.

FIG. 3C is a side elevation drawing of the cranial drill guide of FIG. 3A showing the side opposite to that of FIG. 3B.

FIG. 3D is a proximal-end elevation drawing of the cranial drill guide of FIG. 3A.

FIG. 4A is a perspective drawing of a second alternative embodiment of the inventive cranial drill guide of this invention.

FIG. 4B is a side elevation drawing of the cranial drill guide of FIG. 4A showing a side-opening in the wall of the guide-body.

FIG. 4C is a side elevation drawing of the cranial drill guide of FIG. 4A showing the side opposite to that of FIG. 4B.

FIG. 4D is a proximal-end elevation drawing of the cranial drill guide of FIG. 4A.

FIG. 5A is a perspective drawing of a third alternative embodiment of the inventive cranial drill guide of this invention.

FIG. 5B is a side elevation drawing of the cranial drill guide of FIG. 5A showing a side-opening in the wall of the guide-body.

FIG. 5C is a side elevation drawing of the cranial drill guide of FIG. 5A showing the side opposite to that of FIG. 5B.

FIG. 5D is a proximal-end elevation drawing of the cranial drill guide of FIG. 5A.

FIG. 6A is a perspective drawing of a fourth alternative embodiment of the inventive cranial drill guide of this invention.

FIG. 6B is a side elevation drawing of the cranial drill guide of FIG. 6A showing a side-opening in the wall of the guide-body.

FIG. 6C is a side elevation drawing of the cranial drill guide of FIG. 6A showing the side opposite to that of FIG. 6B.

FIG. 6D is a proximal-end elevation drawing of the cranial drill guide of FIG. 6A.

FIGS. 7A-7C are three views of an embodiment of the inventive cranial drill guide held in a fixture of an exemplary system for positioning instruments during neurosurgical procedures.

### DETAILED DESCRIPTION OF PREFERRED EMBODIMENTS

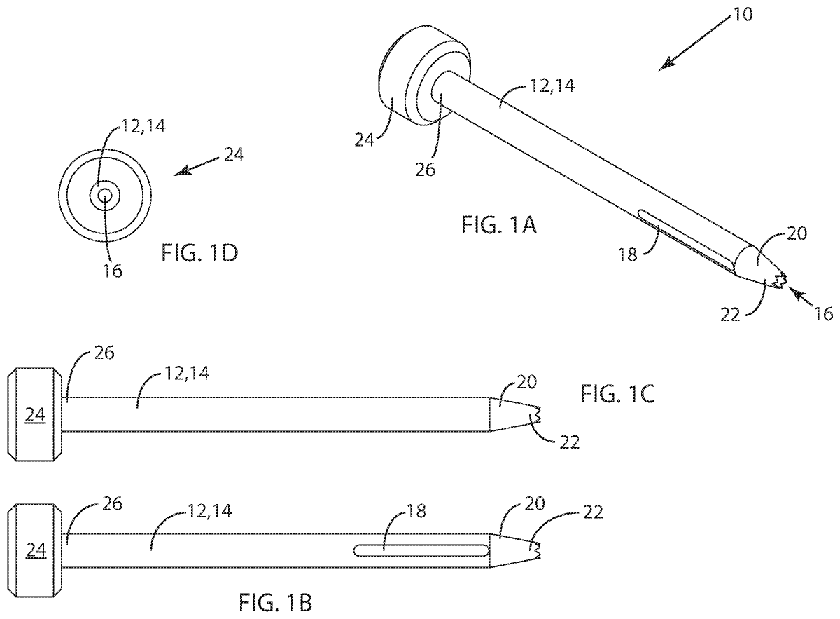

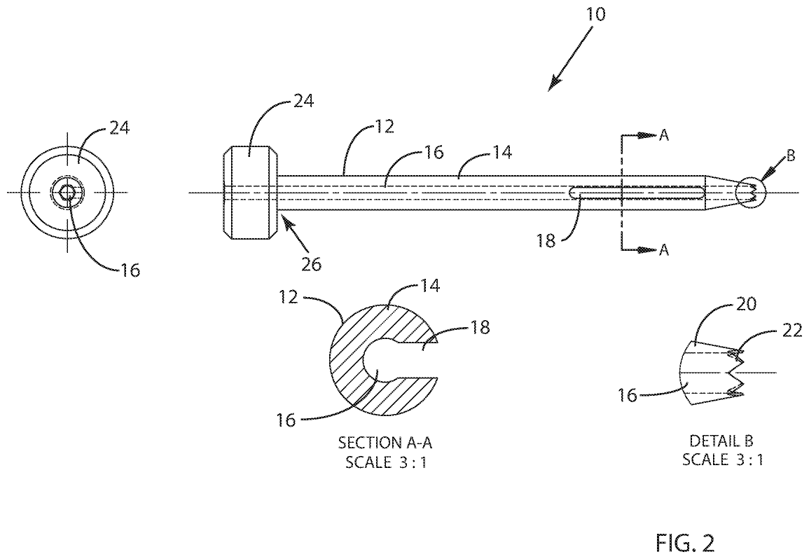

FIG. 1A is a perspective drawing of an embodiment 10 of the inventive cranial drill guide (also identified by reference number 10) of this invention. FIGS. 1B, 1C, and 1D illustrate cranial drill guide 10 in other drawing views. FIGS. 1B and 1C are side elevations from opposite sides of drill 10, and FIG. 1D is a proximal-end elevation drawing of cranial drill guide 10. Also, FIG. 2 is a partial orthographic projection drawing of cranial drill guide 10 showing side and end views and also showing a sectional view and an enlarged view of portions of drill guide 10.

Referring to FIGS. 1A through 2, drill guide 10 includes a guide-body 12 having a wall 14 which defines an internal through-hole 16 along the length of guide-body 12. Through-hole 16 is sized to receive a drill bit for use when drill guide 10 is in position for drilling during a neurosurgical procedure. Wall 14 includes a side-opening 18 positioned near a distal end 20 of drill guide 10. In embodiment 10, side-opening 18 is an elongate slot 18. Side-opening 18 permits bone debris produced during the drilling process to exit from the drill bit to outside drill guide 10 and prevents the drill bit from seizing up and/or generating excessive heat during the drilling process.

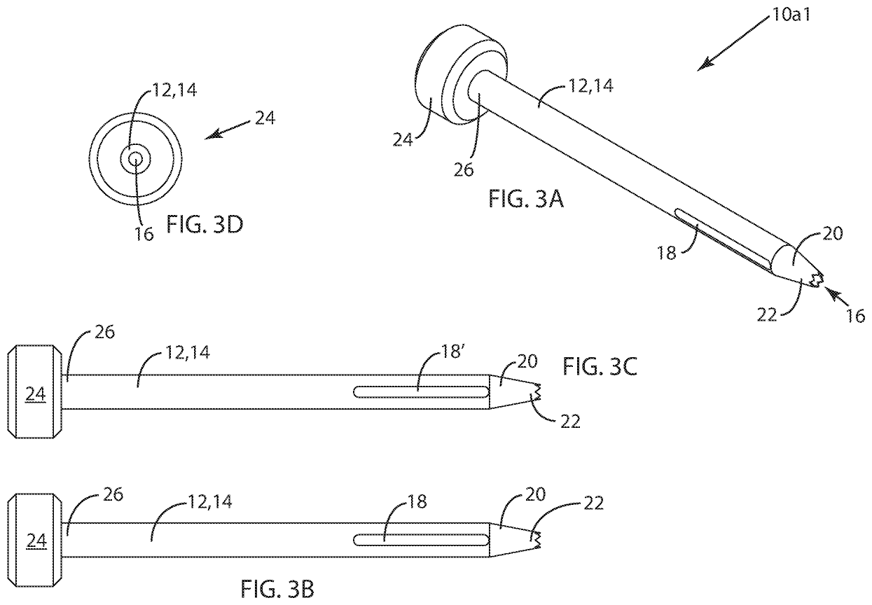

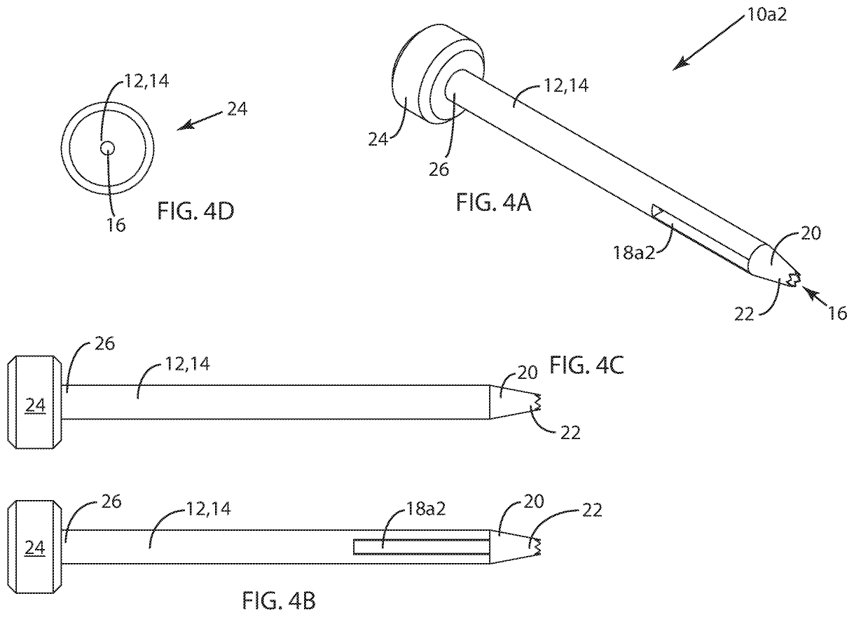

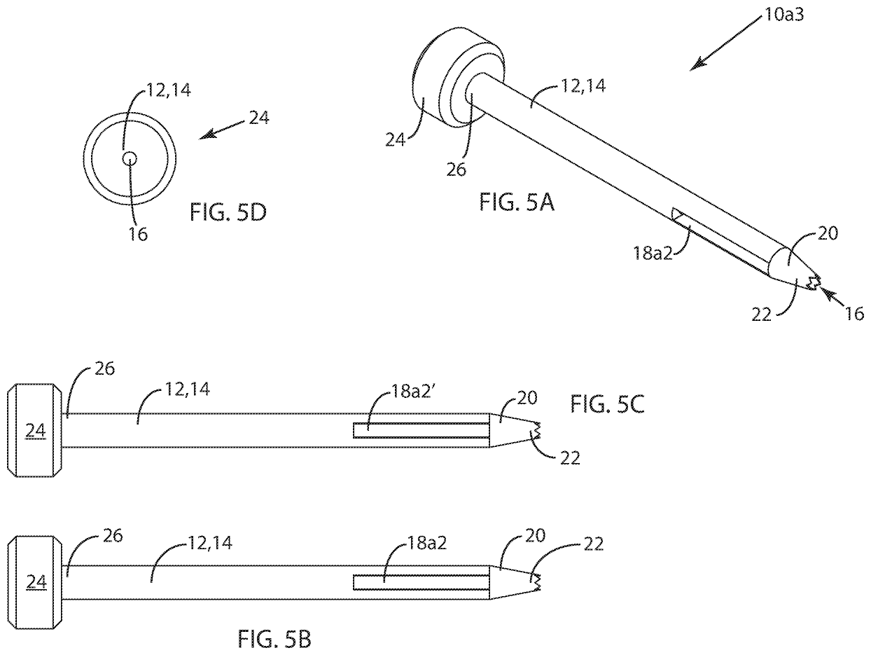

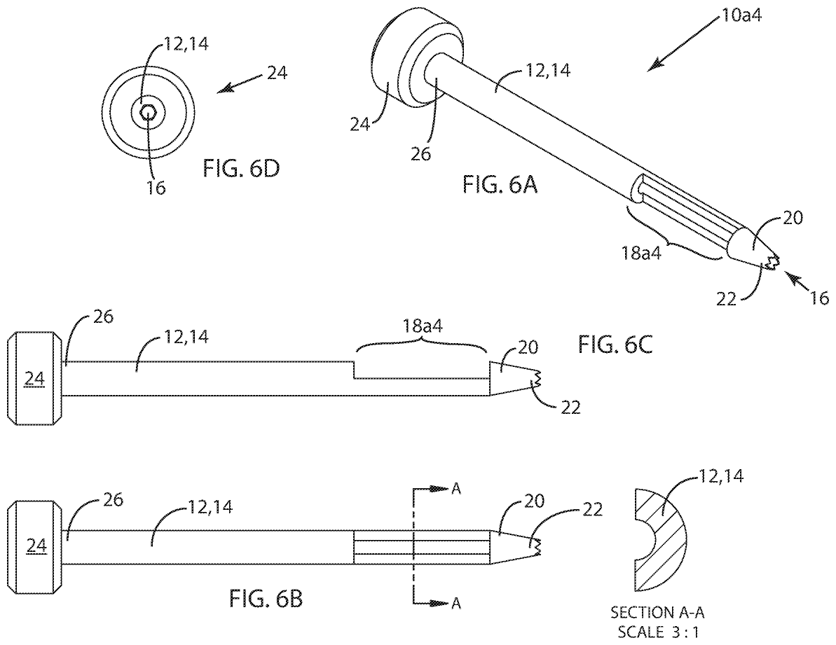

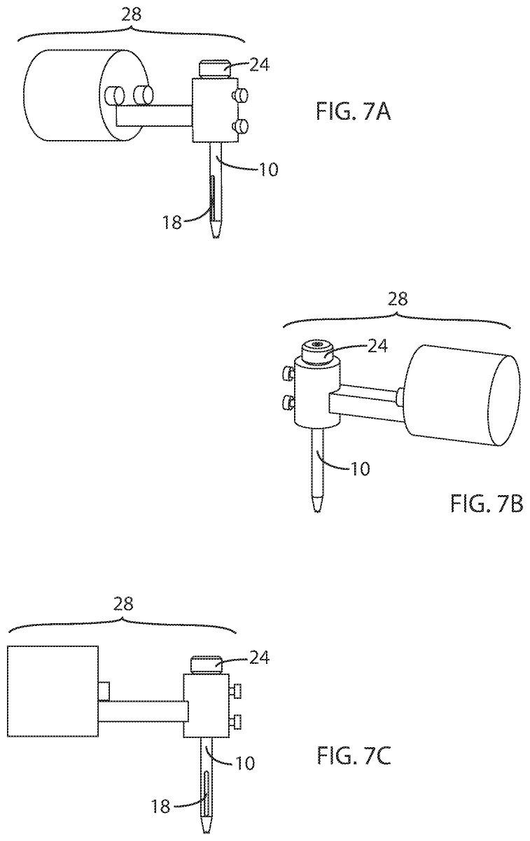

Distal end 20 of cranial drill guide 10 has a serrated tip 22 which secures the position of drill guide 10 with respect to the skull during drilling. Drill guide 10 includes a drill-guide-head 24 at a proximal end 26 of guide 10. Drill-guide-head 24 has cross-sectional and length dimensions different from those of guide-body 12. Drill-guide-head 24 may serve a number of purposes such as fitting into a fixture within systems for positioning instruments during neurosurgical procedures (see FIGS. 7A-7C). FIGS. 3A through 3D illustrate a first alternative embodiment 10a1 of the inventive cranial drill guide (also numbered 10a1); FIGS. 4A through 4D illustrate a second alternative embodiment 10a2 of the inventive cranial drill guide (also numbered 10a2); FIGS. 5A through 5D illustrate a third alternative embodiment 10a3 of the inventive cranial drill guide (also numbered 10a3); and FIGS. 6A through 6D illustrate a fourth alternative embodiment 10a4 of the inventive cranial drill guide (also numbered 10a4). In each of these four alternative embodiments (10a1-10a4), the only structural difference between cranial drill guide 10 and the alternative embodiment is the side-opening or side-openings. All like elements of drill guides 10, 10a1, 10a2, 10a3, and 10a4 have the same reference numbers.

FIGS. 3A-3D, 4A-4D, 5A-5D, and 6A-6D are each similar to FIGS. 1A-1D, respectively. Cranial drill guide 10a1 (FIGS. 3A-3D) includes a second elongate slot 18′ which is positioned opposite to elongate slot 18 so that bone debris can pass out two sides of the guide-body 10a1. Cranial drill guide 10a2 (FIGS. 4A-4D) includes a side-opening 18a2 in place of side-opening 18. Side-opening 18a2 is shaped somewhat differently than side-opening 18 but serves an identical function.

Cranial drill guide 10a3 (FIGS. 5A-5D) is similar to embodiment 10a1 in that it includes a second side-opening 18a2′ opposite to side-opening 18a2, such side-openings 18a2 and 18a2′ serving similar functions to that of side-openings 18 and 18′ in embodiment 10a1.

Cranial drill guide 10a4 (FIGS. 6A-6D) includes a side-opening 18a4 which is formed by removing a portion of wall 14 which is substantially one-half of the cross-section of guide-body 12 along a portion of guide-body 12 to provide a larger side-opening 10a4 for the removal of bone debris from the drill bit.

Alternative embodiments 10a1-10a4 represent just a few of the possible alternative embodiments of side-openings in the cranial drill guide of this invention.

FIGS. 7A through 7C illustrate an exemplary embodiment of cranial drill guide 10 held in a fixture 28 of a system for positioning instruments during neurosurgical procedures. Such systems may include, without limitation, neurosurgical systems such as robots and stereotactic frames.

While the principles of this invention have been described in connection with specific embodiments, it should be understood clearly that these descriptions are made only by way of example and are not intended to limit the scope of the invention.

1. In a cranial drill guide for guiding a drill bit during drilling a brain-access hole in a skull, the improvement comprising an elongate guide-body having a wall defining an internal through-hole therealong, the through-hole sized to receive the drill bit, and a side-opening in the wall for passage of bone debris from the drill bit to outside the guide-body.

2. The cranial drill guide of claim 1 wherein the side-opening is positioned adjacent to a distal end of the drill guide.

3. The cranial drill guide of claim 1 further including a serrated tip at a distal end of the guide-body for securing the position of the drill guide with respect to the skull.

4. The cranial drill guide of claim 3 wherein the side-opening is positioned adjacent to the distal end of the drill guide.

5. The cranial drill guide of claim 1 wherein the side-opening is an elongate slot.

6. The cranial drill guide of claim 1 further including a second side-opening such that bone debris can pass out two sides of the guide-body.

7. The cranial drill guide of claim 6 wherein the side-openings are elongate slots positioned opposite each other.

8. The cranial drill guide of claim 1 wherein the side-opening is formed by removing substantially one-half of the guide-body cross-section along a portion of the guide-body.

9. The cranial drill guide of claim 1 further including a drill-guide-head at a proximal end of the guide-body and having cross-sectional and length dimensions different from those of the guide-body.

10. The cranial drill guide of claim 1 wherein the drill guide is configured to be held within systems for positioning instruments during neurosurgical procedures.

11. The cranial drill guide of claim 1 wherein the guide-body has a circular cross-section.
